(19) United States
(12) Patent Application Publication
(10) Pub. No.: US 2023/0310339 A1
(43) Pub. Date: Oct. 5, 2023

(54) PATCH AND PRODUCTION METHOD THEREOF

(71) Applicant: KANEKA CORPORATION, Osaka (JP)

(72) Inventors: Masaya MIZUTANI, Hyogo (JP); Hiroyuki OGINO, Hyogo (JP); Masaoki GOTO, Hyogo (JP)

(21) Appl. No.: 18/043,764

(22) PCT Filed: Aug. 19, 2021
(86) PCT No.: PCT/JP2021/030285

§ 371 Date: Mar. 2, 2023

(30) Foreign Application Priority Data
Sep. 3, 2020 (JP) 2020-148550
Sep. 3, 2020 (JP) 2020-148551

(51) Int. Cl. A61K 9/70; A61K 31/351

(52) U.S. Cl. CPC A61K 9/7053; A61K 31/351

(57) ABSTRACT

A patch includes an adhesive laver, where the adhesive layer includes (a) 3-[(3R*,4R*)-3-(dimethylamionomethyl)tetrahydropyran-4-yl]phenol and (b) at least one selected from the group consisting of organic acids, amides, and alcohols, and an amount of the (b) is 50 parts by mass or greater relative to 100 parts mass of the (a). A production method of patch includes forming an adhesive layer with a mixture, where the mixture is obtained by mixing (a) 3-[(3R*,4R*)-3-(dimethylamionomethyl)tetrahydropyran-4-yl]phenol, and (b) a polar functional group-containing organic compound at 80° C. or lower.

### TECHNICAL FIELD

The present invention relates to a patch including 3-[(3R*,4R*)-3-(dimethylamionomethyl)tetrahydropyran-4-yl]phenol, and a production method thereof.

### BACKGROUND ART

Although pains are warning signals living bodies release, when the pains are more intense than necessary or becomes chronic, such pains are considered as a symptom to be treated. Most of systemic analgesics developed for treating pains are used as drugs for oral or injection administrations because the systemic analgesics have low skin permeability due to structures and physical properties of the systemic analgesics.

However, drugs for oral administrations have problems such that it is difficult to administer to patients having difficulty in swallowing, such as elderly patients and efficacy of the drug may not last for a sufficient period. The drugs for injection administrations have problems such that an administration of a drug is invasive and requires assistance from a medical practitioner.

Meanwhile, drugs for transdermal administration, particularly patches, have advantages such as administrations of drugs are simple and easy; efficacy of drug is adequately sustained; administrations or drugs are not invasive; and it is easy to pause administrations of drugs. Since it often takes a long period to treat pains, there is a strong need for development of a patch including a drug having efficacy equal to or better than the efficacy of known analgesic.

As a drug that achieves excellent transdermal absorption as well as strong analgesic effects, for example, 3-[(3R*,4R*)-3-(dimethylamionomethyl)tetrahydropyran-4-yl]phenol (may be referred to as “Compound 1” hereinafter) has been known (PTL 1). Moreover, it is disclosed in PTL 1 that Compound 1 is used for a patch, and Compound 1 is highly soluble to a composition used for a patch. Moreover, PTL 1 discloses a formulation of a patch including Compound 1, and a production method of the patch.

### CITATION LIST

### Patent Literature

PTL 1: International Patent Application Publication No. WO 2019/156074

### SUMMARY OF INVENTION

### Technical Problem

As the present inventors produced a patch including 3-[(3R*,4R*)-3-(dimethylamionomethyl)tetrahydropyran-4-yl]phenol according to the method described in PTL 1, crystals of 3-[(3R*,4R*)-3-(dimethylamionomethyl)tetrahydropyran-4-yl]phenol were precipitated and deposited on a release liner. This phenomenon was observed even when a type of the release liner for use was changed.

As a drug is crystalized and deposited on a release liner, a drug content in a patch is reduced, thus the efficacy of the drug is not adequately maintained. When the drug is precipitated to a surface of an adhesive layer of a patch, as well as being deposited on a release liner, crystals of the drug are easily collected from the release liner, which may lead to misuse or abuse of the drug.

According to the formulation of the adhesive layer of the patch disclosed in PTL 1, in addition to that the uniformity of the drug content is insufficient, thus an adequate effect is not be exhibited as a patch, there is a high risk that crystals of the drug deposited on a release liner may be misused or abused.

Moreover, the production method disclosed in PTL 1 is a method where Compound 1, a styrene-isoprene-styrene block copolymer, liquid paraffin, and rosin are melted and mixed, while heating at 150° C., to produce a patch including Compound 1. Considering assurance of safety of production operators, reduction in loads applied to a production device, and inhibition of decomposition of an active ingredient and additives, the production method as described is not a desirable production method of a patch.

The present inventors attempted to produce a medication patch according to the composition disclosed in PTL 1 at a temperature of 80° C. or lower, but 3-[(3R*,4R*)-3-(dimethylamionomethyl)tetrahydropyran-4-yl]phenol did not dissolve in the adhesive layer and a uniformly patch could not be produced.

Accordingly, the present invention aims at solving the above-described various problems existing in the art and achieving the following object. An object of the present invention is to develop a patch in which 3-[(3R*,4R*)-3-(dimethylamionomethyl)tetrahydropyran-4-yl]phenol is uniformly retained in an adhesive layer without being precipitated, and a production method of a patch where 3-[(3R*,4R*)-3-(dimethylamionomethyl)tetrahydropyran-4-yl]phenol is dissolved in an adhesive layer to assure excellent cohesive force and adhesion force, and whereby safety of operators is assured, loads applied to a production device is reduced, and decomposition of an active ingredient and additives is minimized.

### Solution to Problem

The present inventors have diligently conducted research to achieve the above-mentioned object. As a result, the present inventors have the following insights. When a patch includes an adhesive layer where the adhesive layer includes (a) 3-[(3R*,4R*)-3-(dimethylamionomethyl)tetrahydropyran-4-yl]phenol and (b) at least one selected from the group consisting of organic acids, amides, and alcohols and an amount of the (b) is 50 parts by mass or greater relative to 100 parts by mass of the (a), the patch including the adhesive layer in which 3-[(3R*,4R*)-3-(dimethylamionomethyl)tetrahydropyran-4-yl]phenol is uniformly retained without being precipitated can be provided. When a method of producing a patch includes mixing (a) 3-[(3R*,4R*)-3-(dimethylamionomethyl)tetrahydropyran-4-yl]phenol and (b) a polar functional group-containing organic compound at 80° C. or lower to obtain a mixture and forming an adhesive layer with the mixture, the following method of producing a patch can be provided, that is, a production method of a patch where 3-[(3R*,4R*)-3-(dimethylamionomethyl)tetrahydropyran-4-yl]phenol is dissolved in an adhesive layer to assure excellent cohesive force and adhesion force, and whereby safety of operators is assured, load applied to a production device is reduced, and decomposition of an active ingredient and additives is minimized.

The present invention has been accomplished based on the above-described insights of the present inventors, and means for solving the above-described problems are as follows.
<1> A patch including:
   an adhesive layer,
wherein the adhesive layer includes:
   (a) 3-[(3R*,4R*)-3-(dimethylamionomethyl)tetrahydropyran-4-yl]phenol; and
   (b) at least one selected from the group consisting of organic acids, amides, and alcohols, and
wherein an amount of the (b) is 50 parts by mass of greater relative to 100 parts by mass of the (a).
<2> A method of producing a patch according to <1>, the method including:
   mixing 50 parts by mass or greater of (b) at least one selected from the group consisting of organic acids, amides, and alcohols with 100 parts by mass of (a) 3-[(3R*,4R*)-3-(dimethylamionomethyl)tetrahydropyran-4-yl]phenol.
<3> A method of producing a patch, the method including:
   forming an adhesive layer with a mixture,
   wherein the mixture is obtained by mixing 3-[(3R*,4R*)-3-(dimethylamionomethyl)tetrahydropyran-4-yl]phenol and (b) a polar functional group-containing organic compound at 80° C. or lower.

### Advantageous Effects of Invention

The present invention can solve the above-described various problems existing in the art and to achieve the object. The present invention can provide a patch in which 3-[(3R*,4R*)-3-(dimethylamionomethyl)tetrahydropyran-4-yl]phenol is uniformly retained in an adhesive layer without being precipitated, and a production method of a patch where 3-[(3R*,4R*)-3-(dimethylamionomethyl)tetrahydropyran-4-yl]phenol is dissolved in an adhesive layer to assure excellent cohesive force and adhesion force, whereby safety of operators is assured, load applied to a production device is reduced, and decomposition of an active ingredient and additives is minimized.

### DESCRIPTION OF EMBODIMENTS

### Patch

The patch includes an adhesive layer, and may further include other elements.

### Adhesive Layer

The adhesive layer includes (a) 3-[(3R*,4R*)-3-(dimethylamionomethyl)tetrahydropyran-4-yl]phenol and (b) at least one selected from the group consisting of organic acids, amides, and alcohols. The adhesive layer may further include other components.

The adhesive layer is a mixture of the above-mentioned constituent components. An amount of a solvent used for producing the adhesive layer is preferably 0.5% by mass or less, and more preferably 0% by mass, relative to 100% by mass of a total amount of the constituent components of the adhesive layer.

### (a) 3-[(3R*,4R*)-3-(dimethylamionomethyl)tetrahydropyran-4-yl]phenol

A structural formula and synthesis method of the (a) component are as described in Example 1 of International Patent Application Publication No. WO 2019/156074.

Since the (a) component gives a strong pain relief effect, the patch of the present specification can be used for treatment and/or prevention of chronic pains.

An amount of the (a) component in the adhesive layer, i.e., a proportion of the (a) component to 100% by mass of a total amount of constitution components of the adhesive layer, is not particularly limited, and may be appropriately selected in accordance with the intended purpose. Considering uniform retention of the (a) component in the adhesive layer without precipitation, the lower limit of the amount of the (a) component is preferably 0.5% by mass or greater, more preferably 1% by mass or greater, yet more preferably 1.5% by mass or greater, and particularly preferably 2% by mass or greater, and the upper limit of the amount of the (a) component is preferably 30% by mass or less, more preferably 25% by mass or less, yet more preferably 20% by mass or less, and particularly preferably 10% by mass or less.

### (b) At Least One Selected from the Group Consisting of Organic Acids, Amides, and Alcohols

The (b) component is not particularly limited, provided that the (b) component includes at least one selected from the group consisting of organic acids, amides, and alcohols. The (b) component may be appropriately selected in accordance with the intended purpose. The (b) component preferably includes an organic acid because precipitation of crystals can be efficiently reduced.

The (b) component preferably includes at least two selected from the group consisting of organic acids, amides, and alcohols, and more preferably includes organic acids, amides, and alcohols.

### Organic Acids

In the present specification, the term “organic acid” means an organic compound including at least one carboxyl group in each molecule, and may further include a functional group other than the carboxyl group in each molecule.

The organic acids are not particularly limited, and may be appropriately selected in accordance with the intended purpose. Examples of the organic acids include: aliphatic monocarboxylic acids, such as propionic acid, caprylic acid, capric acid, myristic acid, palmitic acid, stearic acid, isostearic acid, behenic acid, Levulinic acid, and the like; aliphatic unsaturated monocarboxylic acids, such as oleic acid, linoleic acid, sorbic acid, and the like; hydroxycarboxylic acids, such as lactic acid (DL-lactic acid, or a mixture of L-lactic acid and/or D-lactic acid and anhydrous lactic acid), gluconic acid, and the like; polyvalent carboxylic acids, such as adipic acid, succinic acid, citric acid, tartaric acid, tumeric acid, maleic acid, malonic acid, malic acid, and the like; aromatic carboxylic acids, such as benzoic acid, salicylic acid, and the like; and sugar derivatives, such as alginic acid, phytic acid and the like. The above-listed examples may be used alone or in combination.

Among the above-listed examples, a fatty acid is preferable, an aliphatic monocarboxylic acid, an aliphatic unsaturated monocarboxylic acid, a hydroxycarboxylic acid, or a polyvalent carboxylic acid is more preferable, capric acid, or isostearic acid, or oleic acid is yet more preferable, capric acid or oleic acid is particularly preferable, and oleic acid is most preferable, because 3-[(3R*,4R*)-3-(dimethylamionomethyl)tetrahydropyran-4-yl]phenol is uniformly retained in the adhesive layer without being precipitated.

An amount of the organic acid is not particularly limited, and may be appropriately selected in accordance with the intended purpose. Considering uniform retention of 3-[(3R*,4R*)-3-(dimethylamionomethyl)tetrahydropyran-4-yl]phenol in the adhesive layer without precipitation, the lower limit of the amount of the organic acid is preferably 50 parts by mass or greater, more preferably 100 parts by mass or greater, yet more preferably 150 parts by mass or greater, yet further more preferably 200 parts by mass or greater, and particularly preferably 300 parts by mass or greater, relative to 100 parts by mass of the (a) component, and the upper limit of the amount of the organic acid is preferably 4,000 parts by mass or less, preferably 3,000 parts by mass or less, and yet more preferably 2,000 parts by mass or less, relative to 100 parts by mass of the (a) component.

A molar equivalent of the organic acid relative to 1 molar equivalent of the (a) component is not particularly limited, and may be appropriately selected in accordance with the intended purpose. Considering uniform retention of 3-[(3R*,4R*)-3-(dimethylamionomethyl)tetrahydropyran-4-yl]phenol in the adhesive layer without precipitation, the lower limit of the molar equivalent of the organic acid relative to 1 molar equivalent of the (a) component is preferably 0.5 molar equivalents or greater, more preferably 1 molar equivalent or greater, yet more preferably 1.5 molar equivalents or greater, and yet further more preferably 2 molar equivalents or greater, and the upper limit of the molar equivalent of the organic acid relative to 1 molar equivalent of the (a) component is 6 molar equivalents or less, more preferably 5 molar equivalents or less, and vet more preferably 3 molar equivalents or less.

### Amides

In the present specification, the term “amides” means organic compounds each including at least one amide group in each molecule. The organic compounds may further include a functional group other than an amide group in each molecule, but organic compounds each including a carboxyl group in each molecule is excluded from the amides.

The amides are not particularly limited, and may be appropriately selected in accordance with the intended purpose. Examples of the amides include: pyrrolidone, such as N-methyl-2-pyrrolidone, lauryl pyrrolidone, 2-pyrrolidone, and the like; aromatic amides, such as acetanilide, crotamiton, nicotinic acid amide, and the like; and aliphatic amides, such as N,N-dimethylacetamide, lauric acid diethanolamide, and the like. The above-listed examples may be used alone or in combination.

Among the above-listed examples, pyrrolidone or an aromatic amide preferable, N-methyl-2-pyrrolidone or crotamiton is more preferable, and crotamiton is yet more preferable, because solubility and dispersibility of 3-[(3R*,4R*)-3-(dimethylamionomethyl)tetrahydropyran-4-yl]phenol are improved.

An amount of the amides is not particularly limited, and may be appropriately selected in accordance with the intended purpose. Considering uniform retention of 3-[(3R*,4R*)-3-(dimethylamionomethyl)tetrahydropyran-4-yl]phenol in the adhesive layer without precipitation, the lower limit of the amount of the amides is preferably 50 parts by mass or greater, more preferably 100 parts by mass or greater, yet more preferably 150 parts by mass or greater, and further more preferably 200 parts by mass or greater, relative to 100 parts by mass of the (a) component, and the upper limit of the amount of the amides is preferably 4,000 parts by mass or less, more preferably 3,000 parts by mass or less, and yet more preferably 2,000 parts by mass or less, relative to 100 parts by mass of the (a) component.

### Alcohols

In the present specification, the term “alcohols” means organic compounds each including at least one hydroxyl group in each molecule. The organic compounds may include a functional group other than the hydroxyl group in each molecule, but organic compounds each including a carboxyl group and/or an amide group in each molecule are excluded from the alcohols.

The alcohols are not particularly limited, and may be appropriately selected in accordance with the intended purpose. Examples or the alcohols include: straight-chain saturated aliphatic alcohols, such as lauryl alcohol, myristyl alcohol, cetanol, cetostearyl alcohol, stearyl alcohol, behenyl alcohol, and the like; branched-chain saturated aliphatic alcohols, such as isostearyl alcohol, hexyldecanol, octyldodecanol, and the like; unsaturated aliphatic alcohol, such as oleyl alcohol, geraniol, and the like; polyhydric alcohols, such as ethylene glycol, propylene glycol, glycerin, 1,3-butanediol, dipropylene glycol, triethylene glycol, 2-ethyl-1,3-hexanediol, 1,2,6-hexanetriol, and the like; polyether-based alcohols, such as polyethylene glycol 200, polyethylene glycol 400, polyoxyethylene(2) ethyl ether (diethylene glycol monoethyl ether), polyoxyethylene(2) lauryl ether, polyoxyethylene(4) lauryl ether, polyoxyethlene (9) lauryl ether, poloxyethylene(2) cetyl ether, polyoxyethylene(2) stearyl ether, ethylene glycol monostearate, and the like; propylene glycol fatty acid monoesters, such as propylene glycol monocaproate, propylene glycol monocaprylate, propylene glycol monocaproate, propylene glycol monolaurate, propylene glycol monomyristate, propylene glycol monostearate, propylene glycol monopalmitostearate, and the like; aromatic alcohols, such as benzyl alcohol, phenylethyl alcohol, phenoxy ethanol, thymol, eugenol, 2-naphthol, vanillin, and the like; and cyclic alcohols, such as it maltol, ethyl maltol, and the like; alkyl lactate compounds, such as ethyl lactate, lauryl lactate, cetyl lactate, and the like; salicylic acid derivatives, such as ethylene glycol salicylate, phenyl salicylate, methyl salicylate, and the like; citric acid derivatives, such as triethyl citrate, tributyl citrate, and the like; terpenes, such as α-terpineol, D-borneol, DL-borneol, L-menthol, DL-menthol, and the like; sorbitans, such as sorbitan monooleate, sorbitan monostearate, sorbitan tristearate, sorbitan monolaurate, sorbitan monopalmitate, sorbitan sesquioleate, sorbitan trioleate, polyoxyethylene sorbitan monolaurate, polyoxyethylene sorbitan monostearate, polyoxyethylene sorbitan trioleate, polyoxyethylene sorbitan tetraoleate, and the like; glycerin derivatives, such as glyceryl monooleate, glyceryl monostearate, glyceryl monomyristate, diglyceryl monooleate, decaglyceryl laurate, α-monoisosteryl glyceryl ether, diglyceryl monoisostearate, diglyceryl monostearate, polyoxyethlene glyceryl monostearate, polyoxyethylene glyceryl triisostearate, polyoxyethylene glyceryl cocoate, and the like; stearic acid derivatives, such as propylene glycol monostearate, ethylene glycol monostearate, and the like; and amines, such as monoethanolamine, diethanolamine, triethnolamine, diisopropanolamine, triisopropanolamine, and the like. The above-listed examples may be used alone or in combination.

Among the above-listed examples, propylene glycol monocaprylate, propylene glycol monolaurate, alkyl lactate, or benzyl alcohol is preferable, propylene glycol monocaprylate, alkyl lactate, or benzyl alcohol is more preferable, propylene glycol monocaprylate or alkyl lactate is yet more preferable, and propylene glycol monocaprylate is particularly preferable, because solubility of 3-[(3R*,4R*)-3-(dimethylamionomethyl)tetrahydropyran-4-yl]phenol is improved.

Examples of a commercial product of propylene glycol monocaprylate include: CAPRYOL™ 90, and CAPRYOL™ PGMC, available from GATTEFOSSE; CAPMUL® PG-8 available from ABITEC; NIKKOL SEFSOL-218 available from NIPPON SURFACTANT INDUSTRIES CO., LTD.; CRODAMOL™ PC available from CRODA, and the like. Examples of a commercial product of propylene glycol monolaurate include: LAUROGLICOL™ 90 and LAUROGLYCOL™ FCC, available from Gattefosse; CAPMUL® PG-12 available from ABITEC, and the like. Examples of a commercial product of alkyl lactate include CERAPRHYL™ 41 and CERAPHYL™ 31, available from Ashland, and the like.

An amount of the alcohols is not particularly limited, and may be appropriately selected in accordance with the intended purpose. Considering uniform re ration of 3-[(3R*,4R*)-3-(dimethylamionomethyl)tetrahydropyran-4-yl]phenol in the adhesive layer without precipitation, the lower limit of the amount of the alcohols is preferably 50 parts by mass or greater, more preferably 100 parts by mass or greater, yet more preferably 150 parts by mass or greater, yet further more preferably 200 parts by mass or greater, and particularly preferably 300 parts by mass or greater, relative to 100 parts by mass of the (a) component, and the upper limit of the amount of the alcohols is preferably 4,000 parts by mass or less, more preferably 3,000 parts by mass or less, and yet more preferably 2,000 parts by mass or less, relative to 100 parts mass of the (a) component.

An amount of the (b) component is not particularly limited, provided that the amount is 50 parts by mass or greater relative to 100 parts by mass of the (a) component. The amount of the (b) component may be appropriately selected in accordance with the intended purpose. Considering uniform retention of 3-[(3R*,4R*)-3-(dimethylamionomethyl)tetrahydropyran-4-yl]phenol in the adhesive layer without precipitation, the lower limit of the amount of the (b) component is preferably 100 parts by mass or greater, more preferably 200 parts by mass or greater, yet more preferably 300 parts by mass or greater, and particularly preferably 500 parts by mass or greater, relative to 100 parts by mass of the (a) component, and the upper limit of the amount of the (b) component is preferably 4,000 parts by mass or less, more preferably 3,000 parts by mass or less, and yet more preferably 2,000 parts by mass or less, relative to 100 parts by mass of the (a) component.

When two or more compounds are included as the (b) component, the amount of the (b) component is a total amount of the compounds included as the (b) component.

### Other Components

The above-mentioned other components are not particularly limited, and may be appropriately selected in accordance with the intended purpose. Examples of the above-mentioned other components include (c) a base polymer, (d) a plasticizer, (e) a tackifier, (f) esters, (g) ethers, (h) an antioxidant, (i) filler, and the like.

### Base Polymer

The base polymer is not particularly limited, and may be appropriately selected in accordance with the intended purpose. Examples of the base polymer include a thermoplastic elastomer, and a thermosetting elastomer.

The thermoplastic elastomer is an elastomer exhibiting thermoplasticity where the elastomer is softened to exhibit fluidity as heated and returns to a rubber-like elastic body as cooled. As the thermoplastic elastomer, various thermoplastic elastomers, such as urethane-based elastomers, acrylic elastomers, styrene-based elastomers, olefin-based elastomer silicone-based elastomers, and the like, are known.

The thermosetting elastomer is an elastomer having relatively high heat resistance without being softened when heated. As the thermosetting elastomer, various thermosetting elastomers, such as acryl-based elastomers, silicone-based elastomers, natural rubber, and the like, are known.

Among the above-listed examples, a thermoplastic elastomer is preferable, a styrene-based thermoplastic elastomer is more preferable, and a styrene-based block copolymer is yet more preferable, considering improvement in production efficiency of the adhesive layer (a blending efficiency improves as the thermoplastic elastomer softened to exhibit fluidity when heated) and a capability tri be blended with additives, such as the (b) component, the (d) component, and the like, without impairing adhesion force or cohesive force of the adhesive layer.

The styrene-based block copolymer is not particularly limited, and may be appropriately selected in accordance with the intended purpose. Examples of the styrene-based block copolymer include a styrene-butadiene block copolymer, a styrene-butadiene-styrene block copolymer, a styrene-isoprene block copolymer, a styrene-isoprene-styrene block copolymer, a styrene-ethylene/butylene block copolymer, a styrene-ethylene/butylene-styrene block copolymer, a styrene-ethylene/propylene block copolymer, a styrene-ethylene/propylene-styrene block copolymer, a styrene-isobutylene block copolymer, a styrene-isobutylene-styrene block copolymer, and the like.

The term “ethylene/butylene” means a copolymer block of ethylene and butylene, and the term “ethylene/propylene” means a copolymer block of ethylene and propylene.

The above-listed styrene-based block copolymers may be used alone or in combination.

Among the above-listed styrene-based block copolymer, at least one selected from the group consisting of a styrene-isoprene-styrene block copolymer and a styrene-isoprene block copolymer is particularly preferably used considering both adequate adhesion on skin and reduction adhesive residues due to improvement in cohesive force of the adhesive layer, as well as availability and easy handling thereof for patches. The styrene-based block copolymer is most preferably a mixture of a styrene-isoprene-styrene block copolymer and a styrene-isoprene block copolymer.

When a mixture of a styrene-isoprene-styrene block copolymer and a styrene-isoprene block copolymer is used as the styrene-based block copolymer, an amount of the styrene-isoprene block copolymer in the mixture is preferably 15% by mass or greater, more preferably 20% by mass or greater, yet more preferably 30% by mass or greater, yet further more preferably 40% mass or greater, and particularly preferably 50% by mass or greater. The upper limit of the amount of the styrene-isoprene block copolymer in the mixture is preferably 80% by mass or less.

In the styrene-isoprene-styrene block copolymer, an amount of the styrene is preferably 5% by mass or greater and 60% by mass or less, and more preferably 10% by mass or greater and 50% by mass or less. Moreover, a weight average molecular weight of the styrene-isoprene-styrene block copolymer measured by gel permeation chromatography (GPC) is preferably 20,000 or greater and 500,000 or less, and more preferably 30,000 or greater and 300,000 or less.

In the styrene-isoprene block copolymer, an amount of styrene is preferably 5% by mass or greater and 50% by mass or less, and more preferably 10% by mass or greater and 40% by mass or less. Moreover, a weight average molecular weight of the styrene-isoprene block copolymer as measured by GPC is preferably 10,000 or greater and 500,000 or less, and more preferably 20,000 or greater and 300,000 or less.

As the styrene-isoprene-styrene block copolymer and the styrene-isoprene block copolymer, copolymers produced by any of methods known in the art may be used. Moreover, commercial products satisfying the above-described properties may be used as the styrene-isoprene-styrene block copolymer and the styrene-isoprene block copolymer. Moreover, a mixture of the styrene-isoprene-styrene block copolymer and the styrene-isoprene block copolymer is also available as commercial products. Commercial products of the mixture in which the styrene-isoprene-styrene block copolymer satisfying the above-described properties and the styrene-isoprene block copolymer satisfying the above-described properties are blended at the above-mentioned blending ratio are suitably used.

Examples of the commercial products include: KRATON® D1111, KRATON® D1163, KRATON® D1113, and KRATON® D1119, available from KRATON POLYMERS; JRS SIS® 5229, JRS SIS® 5002, JRS SIS® 5403, and JRS SIS® 5505, available from JSR; and Quintac® 3421, Quintac® 3433N, Quintac® 3520, Quintac® 3450, and Quintac 3270, available from Zeon Corporation.

Among the above-listed examples, KRATON® D1163, KRATON® D1113, JSR SIS® 5403, SIS® 5505, Quintac® 3433N, or Quintac® 3520 is preferable, and JSR SIS® 5505 or Quintac® 3520 is more preferable, considering a blending ratio between the triblock copolymer and the diblock copolymer, and viscosity of a solution. The above-lasted styrene-based block copolymers are each a mixture of a styrene-isoprene-styrene block copolymer and a styrene-isoprene block copolymer, where an amount of the styrene-isoprene block copolymer in the mixture is 50% by mass or greater.

An amount of the base polymer in the adhesive layer, i.e., a proportion of the base polymer to 100% by mass of a total amount of constituent components of the adhesive layer, is not particularly limited, and may be appropriately selected in accordance with the intended purpose. Considering shape retention of the adhesive layer and adhesion on skin, the lower limit of the amount of the base polymer is preferably 10% by mass or greater, more preferably 15% by mass or greater, and yet more preferably 20% by mass or greater, and the upper limit of the amount of the base polymer is preferably 50% by mass or less, more preferably 40% by mass or less, and yet more preferably 35% by mass or less.

### (d) Plasticizer

The plasticizer is not particularly limited, and may be appropriately selected in accordance with the intended purpose. Examples of the plasticizer include liquid paraffin, squalene, squalane, pristane, hexadecyl isostearate, octyldodecyl myristate, and the like. The above-listed examples may be used alone or in combination.

Among the above-listed examples, liquid paraffin, hexadecyl isostearate, or octyldodecyl myristate is preferable, and octyldodecyl myristate is more preferable.

The liquid paraffin is colorless and odorless, and a mixture of fluid saturated hydrocarbons. As liquid paraffin, liquid paraffin meeting the standard specified by Japanese Pharmacopoeia, US Pharmacopoeia, etc. is suitably used.

Among the above-listed examples, liquid paraffin having a high viscosity is preferable considering adhesion.

Specifically, the liquid paraffin having a kinematic viscosity of 60 mm²/s or greater at 40° C. is preferred, more preferably 70 mm²/s or greater, and more preferably 30 mm²/s or greater.

The upper limit of the kinematic viscosity is not particularly limited. Considering easiness of handling and availability, for example, the upper limit of the kinematic viscosity is preferably 500 mm²/s or less, and more preferably 250 mm²/s or less.

The “kinematic viscosity” a value obtained by converting a viscosity (mPa·s) measured according to “Method II Viscosity measurement by rotational viscometer (2.12 Single cylinder-type rotational viscometer (Brookfield type viscometer)” in “2.53 Viscosity Determination” of General Tests in “Japanese Pharmacopoeia 17th Edition” into a kinematic viscosity.

An amount of the plasticizer in the adhesive laver, i.e., a proportion of the plasticizer to 100% by mass of a total amount of the constituent components of the adhesive layer is not particularly limited, and may be appropriately selected in accordance with the intended purpose. The lower limit is, for example, preferably 1% by mass or greater, more preferably 2% by mass or greater, and yet more preferably 3% by mass or greater. The upper limit is, for example, preferably 60% by mass or less, more preferably 55% by mass or less, and yet more preferably 50% by mass or less.

### (e) Tackifier

The tackifier is a tackifier generally used in a typical field of patches. Examples of the tackifier include a rosin-based resin, a polyterpene-based resin, a coumarone-indene resin, a petroleum resin, a terpene resin, a terpene-phenol resin, an alicyclic saturated hydrocarbon resin, and the like. The above-listed examples may be used alone or in combination.

Among the above-listed examples, a rosin-based resin, a terpene resin, or an alicyclic saturated hydrocarbon resin is preferred.

The rosin-based resin is not particularly limited, and may be appropriately selected in accordance with the intended purpose. Examples of the rosin-based resin include rosin esters.

An amount of the tackifier in the adhesive layer, i.e., a proportion of the tackifier to 100% by mass of a total amount of the constituent components of the adhesive layer, is not particularly limited, and may be appropriately selected in accordance with the intended purpose. The lower limit of the amount of the tackifier is preferably 5% by mass or greater, more preferably 10% by mass or greater, and yet more preferably 15% by mass or greater. The upper limit of the amount of the tackifier is preferably 40% by mass or less, more preferably 35% mass or less, and yet more preferably 30% by mass or less.

### (f) Esters

In the present specification, the term “esters” mean organic compounds each including at least one ester group in each molecule, and organic compounds each including a carboxyl group and/or a hydroxyl group and/or an amide group in each molecule are excluded from the esters.

The esters are not particularly limited, and may be appropriately selected in accordance with the intended purpose. Examples of the esters include: esters between fatty acids and monovalent aliphatic alcohols, such as isoamyl isovalerate, isostearyl palmitate, cetyl 2-ethylhexanate (cetyl isooctanoate), ethyl oleate, decyl oleate, isopropyl palmitate, cetyl palmitate, isopropyl myristate, cetyl myristate, myristyl myristate, batyl monostearate, hexyl laurate, methyl laurate, isopropyl linoleate, ethyl linoleate, cocoyl caprylocaprate, and the like; diesters, such as diisopropyl adipate, diisobutyl adipate, diisopropyl sebacate, diethyl sebacate, and the like; propylene glycol-based diesters, such as propylene glycol dicaprylate, propylene glycol dicaprate, propylene glycol dicaprylocaprate, propylene glycol diacetate, and the like; glycerin-based esters, such as triacetin, tricaprylin (glycerol trioctanoate), caprylic/capric triglyceride, glyceryl trioctanoate (triethylhexanoin), triglycerol diisostearate, medium-chain triglycerides, and the like; citric acid-based esters, such, as triethyl O-acetylcitrate, tributyl O-acetylcitrate, and the like; cyclic carbonates, such as ethylene carbonate, propylene carbonate, maleic anhydride, and the like; and aromatic esters, such as benzyl benzoate, benzyl acetate, diethyl phthalate, dibutyl phthalate, butyl phthalyl butyl glycolate, and the like.

### (g) Ethers

In the present specification, the term “ethers” means organic compounds each including at least one ether group in each molecule, and organic compounds each including a carboxyl group and/or a hydroxyl group and/or an amide group in each molecule are excluded from the ethers.

The ethers are not particularly limited, and may be appropriately selected in accordance with the intended purpose. Examples of the ethers include dimethyl isosorbide, piperonyl butoxide, and the like.

### (h) Antioxidant

The antioxidant is not particularly limited, and may be appropriately selected in accordance with the intended purpose. Examples of the antioxidant include: phenols, such as 2,6-di-tert-butyl-hydroxytoluene (BHT), butylated hydroxyanisole (BHA), propyl gallate, hydroquinone, oxybenzone, pentaerythritol-tetrakis[3-(3,5-di-tert-butyl-4-hydroxyphenyl)propipnate], rutin, catechin, and the like; triazoles, such as 1,2,3-benzotriazole and the like; sulfur-containing compounds, such as sulfur, L-cysteine, L-cystine, DL-methionine, L-methionine, alpha-thioglycerol, allyl isothiocyanate, thioglycolic acid, sodium thioglycolate, potassium thiocyanate, sodium thiosulfate, sodium thiomalate, 2-mercaptobenzimidazole, thiourea, N,N-dimethylthiourea, and the like; amines, such as EDTA-2Na and the like; and vitamins, such as tocopherol, tocopherol acetate, ascorbic acid, isoascorbic acid, ascorbyl stearate, ascorbyl palmitate, erythorbic acid, hesperidin, methyl hesperidin, riboflavin, riboflavin butyrate, and the like. The above-listed examples may be used alone or in combination.

Among the above-listed examples, dibutyl hydroxytoulene or 2-mercaptobenzimidazole is preferred.

### (i) Filler

The filler is not particularly limited, and may be appropriately selected in accordance with the intended purpose. Examples of the filler include: silicon compounds, such as silicic anhydride, light anhydrous silicic acid, silicic acid hydrate, and the like; cellulose derivatives, such as ethyl cellulose, methyl cellulose, hydroxypropyl cellulose, hydroxypropyl methyl cellulose, and the like; water-soluble polymers, such as polyvinyl alcohol and the like; aluminum compounds, such as dried aluminum hydroxide gel, hydrous aluminum silicate, and the like; kaolin; titanium oxide, and the like. As the filler, any of the foregoing compounds may be used alone, or two or more of the foregoing compounds may be used in combination.

### Other Elements

The above-mentioned other elements are not particularly limited, and may be appropriately selected in accordance with the intended purpose. Examples of the above-mentioned other elements include a support, a release liner, and the like. In other words, the patch of the present invention may include a support, an adhesive layer, and a release liner stacked in this order.

The support is not particularly limited, and may be appropriately selected in accordance with the intended purpose. Examples or the support include supports generally used for adhesive sheets to adhere to skin or for transdermal drugs.

A material of the support is not particularly limited, and may be appropriately selected in accordance with the intended purpose. Examples of the material include polyesters (e.g., polyethylene terephthalate and the like), polyolefins (e.g., polyethylene, polypropylene, and the like), polyurethanes, ethylene-vinyl acetate copolymers, polyvinyl chloride, and the like.

A structure of the support may be a single-layer structure or a multiple-layer structure. Moreover, the support may be of knitted fabrics, woven fabrics, nonwoven fabrics, a film, foam, porous body, a network structure, a sheet, or a flat plate.

In order to minimize accumulation of static electricity within the support, an antistatic agent may be added to the woven fabrics, nonwoven fabrics, film, or the like constituting the support. Moreover, a laminate of a film with nonwoven fabrics or woven fabrics, or a laminate of a film with nonwoven fabrics and woven fabrics may be used as the support to assure excellent anchoring to an adhesive layer.

A thickness of the support is not particularly limited, and may be appropriately selected in accordance with the intended purpose. For a film serving as the support, the lower limit of the thickness of the support is, for example, preferably 10 μm or greater, more preferably 15 μm or greater, and the upper limit of the thickness of the support is, for example, preferably 100 μm or less, more preferably 50 μm or less. For a porous sheet serving as the support, such as a woven fabric support, a nonwoven fabric support, and a foam support, the lower limit of the thickness of the support is preferably 50 μm or greater, more preferably 100 μm or greater, and the upper limit of the thickness of the support is preferably 2,000 μm or less, more preferably 1,000 μm or less.

The release liner is not particularly limited, and may be appropriately selected in accordance with the intended purpose. Examples of the release liner include: resin films, such as glassine, polyolefins (e.g., polyethylene, polypropylene, and the like), polyesters (e.g., polyethylene terephthalate and the like), polystyrenes, and the like; aluminum films; polyethylene foam films or polypropylene foam films; and laminates each including two or more of the foregoing ones. Moreover, the release liner subjected to silicone processing, fluororesin processing, embossing, hydrophilic processing, or hydrophobic processing may be used.

A thickness of the release liner is not particularly limited, and may be appropriately selected in accordance with the intended purpose. The lower limit of the thickness of the release liner preferably 10 μm or greater, more preferably 15 μm or greater, and the upper of the thickness of the release liner is (preferably 200 μm or less, more preferably 150 μm or less.

### Production Method of Patch: First Embodiment

In a first embodiment, the production method of a patch is a method of producing the above-described patch. The production method includes a mixing step, which includes mixing 50 parts by mass or greater of (b) at least one selected from the group consisting of organic acids, amides, and alcohols with 100 parts by mass of (a) 3-[(3R*,4R*)-3-(dimethylamionomethyl)tetrahydropyran-4-yl]phenol. The production method may further include other steps.

The (a) 3-[(3R*,4R*)-3-(dimethylamionomethyl)tetrahydropyran-4-yl]phenol and the (b) at least one selected from the group consisting of organic acids, amides, and alcohols are as described in the (Patch).

### Mixing Step

The mixing step is not particularly limited, and may be appropriately selected in accordance with the intended purpose. The mixing step is preferably mixing in the presence of a volatile solvent.

The volatile solvent is not particularly limited, and may be appropriately selected in accordance with the intended purpose. Examples of volatile solvent include aromatic hydrocarbons (e.g., toluene and the like), alicyclic hydrocarbons (e.g., cyclohexane, methyl cyclohexane, and the like), aliphatic hydrocarbons (e.g. hexane, heptane, and the like), ethers (e.g., tetrahydrofuran, diethyl t-butyl methyl ether, and the like), ketones (e.g., acetone, methyl ethyl ketone, methyl isobutyl ketone, and the like), alcohols (e.g., ethanol, propanol, butanol, and the like), acetic acid esters (e.g., ethyl acetate, propyl acetate, isopropyl acetate, butyl acetate, isobutyl acetate, and the like), and the like. The above-listed examples may be used alone or in combination.

Considering excellent solubility of components constituting the adhesive layer, a single solvent or a mixture of two or more solvents selected from aromatic hydrocarbons (e.g., toluene and the like), alicyclic hydrocarbons (e.g., cyclohexane, methyl cyclohexane, and the like), and aliphatic hydrocarbons (e.g., hexane, heptane, and the like) is preferably used.

A mixing method in the mixing step is not particularly limited provided that the mixing method is a method of mixing the (a) 3-[(3R*,4R*)-3-(dimethylamionomethyl)tetrahydropyran-4-yl]phenol and the (b) at least one selected from the group consisting of organic acids, amides, and alcohols. The mixing method may be appropriately selected in accordance with the intended purpose.

Among the above-listed examples, a step including mixing the base polymer with a mixture obtained by mixing (a) 3-[(3R*,4R*)-3-(dimethylamionomethyl)tetrahydropyran-4-yl]phenol and (b) at least one selected from the group consisting of organic acids, amides, and alcohols is preferred, and a step including mixing the plasticizer with a mixture obtained by mixing (a) 3-[(3R*,4R*)-3-(dimethylamionomethyl)tetrahydropyran-4-yl]phenol and (b) at least one selected from the group consisting of organic acids, amides, and alcohols, followed by mixing the resulting mixture with the base polymer is more preferred considering dissolution efficiency.

When (b) at last two selected from the group consisting organic acids, amides, and alcohols are used, the selected compounds are preferably added at once.

A temperature for mixing the (a) 3-[(3R*,4R*)-3-(dimethylamionomethyl)tetrahydropyran-4-yl]phenol and the (b) at least one selected from the group consisting of organic acids, amides, and alcohols is not particularly limited, and may be appropriately selected in accordance with the intended purpose. The temperature is preferably 80° C. or lower, more preferably 70° C. or lower, yet more preferably 60° C. or lower, particularly preferably 50° C. or lower, and most preferably 40° C. or lower.

The temperature for mixing the (a) component and the (b) component being 80° C. or lower means that an inner temperature (an internal temperature) of the mixture is 80° C. or lower. When the mixture is heated using an external device such as an oil bath, a hot plate, and a mixer equipped with a thermal control function, the temperature is not particularly limited as long as the temperature of the mixture itself is 80° C. or lower. Specifically, there is no problem even if a set temperature of the external device is higher than 80° C.

A temperature for mixing the thermoplastic elastomer with a mixture obtained by mixing the (a) 3-[(3R*,4R*)-3-(dimethylamionomethyl)tetrahydropyran-4-yl]phenol and the (b) at least one selected from the group consisting of organic acids, amides, and alcohols is not particularly limited, and may be appropriately selected in accordance with the intended purpose. The temperature is preferably 80° C. or lower, more preferably 70° C. or lower, yet more preferably 60° C. or lower, particularly preferably 50° C. or lower, and most preferably 40° C. or lower.

The temperature for mixing the base polymer with the mixture obtained by mixing the (a) component and the (b) component is as described above, and there is no problem even if a set temperature of an external device is greater than 80° C.

A temperature for mixing the plasticizer with the mixture obtained by mixing the (a) 3-[(3R*,4R*)-3-(dimethylamionomethyl)tetrahydropyran-4-yl]phenol and the (b) at least one selected from the group consisting of organic acids, amides, and alcohols is not particularly limited, and may be appropriately selected in accordance with the intended purpose. The temperature is preferably 80° C. or lower, more preferably 70° C. or lower, yet more preferably 60° C. or lower, particularly preferably 50° C. or lower, and most preferably 40° C. or lower. A temperature for mixing the resulting mixture with the thermoplastic elastomer is not particularly limited, and may be appropriately selected in accordance with the intended purpose. The temperature is preferably 80° C. or lower, more preferably 70° C. or lower, yet more preferably 60° C. or lower, particularly preferably 50° C. or lower, and most preferably 40° C. or lower.

The temperature for mixing the plasticizer with the mixture obtained by the (a) component and the (b) component is also as described above, and there is no problem even if a set temperature of an external device is higher than 80° C.

### Other Steps

The above-mentioned other steps are not particularly limited, and may be appropriately selected in accordance with the intended purpose. Examples of the above-mentioned other steps include a coating step after the mixing step, a step of laminating a support and the adhesive layer, and a step of laminating a release liner.

The support, the adhesive layer, and the release liner are as described in the (Patch).

### Coating Step after Mixing Step

The coating of the coating liquid for forming an adhesive layer, which is obtained in the mixing step, is performed by applying the coating liquid to the support or the release liner by means of a commonly used coater, such as a roll coater, a die coater, a gravure roll coater, a reverse roll coater, a kiss roll coater, a dip roll coater, a bar coater, a knife coater, and a spray coater.

Drying of the coating liquid is performed with heating, for example, preferably at a temperature of 40° C. or higher, and preferably at a temperature of 150° C. or lower, more preferably 100° C. or lower, yet more preferably or lower, and particularly preferably 80° C. or lower. Among the above-listed examples, the heating is preferably performed at a temperature of 40° C. or higher and 150° C. or lower, more preferably 40° C. or higher and 100° C. or lower, yet more preferably 40° C. or higher and 90° C. or lower, and particularly preferably 40° C. or higher and 80° C. or lower. The drying temperature, drying duration, and drying method are adjusted in accordance with a solvent to be used or an amount of the solvent used.

### Step of Laminating Support and Adhesive Layer

The step of laminating a support and the adhesive layer is not particularly limited, and may be appropriately selected in accordance with the intended purpose. Examples of the step of laminating the support and the adhesive layer include a method where the support is bonded to the adhesive layer through pressure bonding to laminate the support and the adhesive layer.

### Step of Laminating Release Liner

The step of laminating a release liner is not particularly limited, and may be appropriately selected in accordance with the intended purpose. Examples of the step of laminating a release liner include: a step including, before the step of laminating the support and the adhesive layer, spreading the coating liquid for forming an adhesive layer on a release liner and drying a solvent in the coating liquid to laminate an adhesive layer on a surface of the release liner (spreading and drying); a step where, after the step of laminating the support and the adhesive layer, bonding the release liner to the adhesive layer through pressure bonding to laminate the adhesive layer and the release liner; and the like.

### Production Method of Patch: Second Embodiment

In a second embodiment, the production method of a patch includes a step of forming an adhesive layer and may further include other steps. The step of forming the adhesive layer includes forming an adhesive layer using a mixture obtained by mixing (a) 3-[(3R*,4R*)-3-(dimethylamionomethyl)tetrahydropyran-4-yl]phenol and (b) a polar functional group-containing organic compound at 80° C. or lower. The production method may further include other steps.

### Step of Forming Adhesive Layer

The step of forming the adhesive layer is a step including forming an adhesive layer using a mixture obtained by mixing (a) 3-[(3R*,4R*)-3-(dimethylamionomethyl)tetrahydropyran-4-yl]phenol and (b) a polar functional group-containing organic compound.

In the step of forming the adhesive layer, a mixture obtained by mixing at least (a) 3-[(3R*,4R*)-3-(dimethylamionomethyl)tetrahydropyran-4-yl]phenol and (b) a polar functional group-containing organic compound is used, but a mixture obtained by further mixing with (c) other components may be used.

### (a) 3-[(3R*,4R*)-3-(dimethylamionomethyl)tetrahydropyran-4-yl]phenol

A structural formula and synthesis method of the (a) component are as described in Example 1 of International Patent Application Publication No. WO 2019/156074.

Since the (a) component exhibits strong analgesic effects, the patch produced by the production method of a patch of the present disclosure can be used for treatments and/or preventions of chronic pains.

An amount of the (a) component in the adhesive layer, i.e., a proportion of the (a) component to 100% by mass of a total amount of constituent components of the adhesive layer, is not particularly limited, and may be appropriately selected in accordance with the intended purpose. Considering uniform retention of 3-[(3R*,4R*)-3-(dimethylamionomethyl)tetrahydropyran-4-yl]phenol in the adhesive layer without precipitation, the lower limit of the amount of the (a) component is preferably 0.5% by mass or greater, more preferably 1% by mass or greater, yet more preferably 1.5% by mass or greater, and particularly preferably 2% by mass or greater, and the upper limit of the amount of the (a) component is preferably 30% by mass or less, more preferably 25% by mass or less, yet more preferably 20% by mass or less, and particularly preferably 10% by mass or less.

### (b) Polar Functional Group-Containing Organic Compound

The polar functional group-containing organic compound is not particularly limited, and may be appropriately selected in accordance with the intended purpose. Examples of the polar functional group-containing organic compound include organic acids, amides, alcohols, esters, ethers, and the like.

Among the above-listed examples, the polar functional group-containing organic compound preferably includes at least one selected from the group consisting of organic acids, amides, and alcohols, more preferably at least two selected from the group consisting of organic acids, amides, and alcohols, and yet more preferably organic acids, amides, and alcohols.

The organic acids, the amides, the alcohols, the esters and the ethers are as described in the section of (Patch).

An amount of the (b) component is not particularly limited, and may be appropriately selected in accordance with the intended purpose. The lower limit of the amount of the (b) component is preferably 50 parts by mass or greater, more preferably 100 parts by mass or greater, yet more preferably 200 parts by mass or greater, particularly preferably 300 parts by mass or greater, and most preferably 500 parts by mass or greater, relative to 100 parts by mass of the (a) component, and the upper limit of the amount of the (b) component is preferably 4,000 parts by mass or less, more preferably 3,000 parts by mass, and yet more preferably 2,000 parts by mass or less, relative to 100 parts by mass of the (a) component.

When two or more compounds are included as the (b) component, the amount of the (b) component is a total amount of the compounds included as the (b) component.

### (c) Other Components

The above-mentioned other components are not particularly limited, and may be appropriate selected in accordance with the intended purpose. Examples of the above-mentioned other components include a base polymer, a plasticizer, a tackifier, an antioxidant, filler, and the like.

The plasticizer, the tackifier, the antioxidant, and the filler are as described in the (Patch).

### Base Polymer

The base polymer is not particularly limited, and may be appropriately selected in accordance with the intended purpose. Examples of the base polymer include a thermoplastic elastomer and a thermosetting elastomer.

The thermoplastic elastomer an elastomer exhibiting thermoplasticity where the elastomer is softened to exhibit fluidity as heated and returns to a rubber-like elastic body as cooled. As the thermoplastic elastomer, various thermoplastic elastomers, such as urethane-based elastomers, acrylic elastomers, styrene-based elastomers, olefin-based elastomers, silicone-based elastomers, and the like, are known.

The thermosetting elastomer is an elastomer having relatively high heat resistance without being softened when heated. As the thermosetting elastomer, various thermosetting elastomers, such as acryl-based elastomers, silicone-based elastomers, natural rubber, and the like, are known.

Among the above-listed examples, a thermoplastic elastomer is preferable, a styrene-based thermoplastic elastomer is more preferable, and a styrene-based block copolymer is yet more preferable, considering improvement in production efficiency of the adhesive layer (blending efficiency improves as the thermoplastic elastomer is softened to exhibit fluidity when heated) and a capability to be blended with the (b) component and additives, such as the plasticizer, without significantly impairing adhesion force or cohesive force of the adhesive layer.

The styrene-based block copolymer is as described in the section of (Patch) above.

An amount of the base polymer in the adhesive layer, i.e., a proportion of the base polymer to 100% by mass of a total amount of constituent components of the adhesive layer, is not particularly limited, and may be appropriately selected in accordance with the intended purpose. Considering shape retention of the adhesive layer and adhesion on skin, the amount of the base polymer is preferably 10% by mass or greater and 50% by mass or less, more preferably 15% by mass or greater and 40% by mass or less, and yet more preferably 20% by mass or greater and 35% by mass or less.

### Adhesive Layer Formation Method

The adhesive der formation method is not particularly limited, provided that the formation method is a method where a mixture obtained by mixing (a) 3-[(3R*,4R*)-3-(dimethylamionomethyl)tetrahydropyran-4-yl]phenol and (b) a polar functional group-containing organic compound is used to form an adhesive layer. The formation method may be appropriately selected in accordance with the intended purpose.

Among the above-listed examples, considering dissolution efficiency, a step where the base polymer is mixed with a mixture obtained by mixing (a) 3-[(3R*,4R*)-3-(dimethylamionomethyl)tetrahydropyran-4-yl]phenol and (b) a polar functional group-containing organic compound to form an adhesive layer is preferred, and a step where the plasticizer is mixed with a mixture obtained by mixing (a) 3-[(3R*,4R*)-3-(dimethylamionomethyl)tetrahydropyran-4-yl]phenol and (b) a polar functional group-containing organic compound, followed by mixing the resulting mixture with the base polymer, to form an adhesive layer is more preferred.

When two or more kinds of the (b) polar functional group-containing organic compounds are used, all the (b) polar functional group-containing organic compounds are preferably added at once.

A temperature for mixing the (a) 3-[(3R*,4R*)-3-(dimethylamionomethyl)tetrahydropyran-4-yl]phenol and the (b) polar functional group-containing organic compound is not particularly limited, provided that the temperature is 80° C. or lower. The temperature may be appropriately selected in accordance with the intended purpose. The temperature is preferably 70° C. or lower, more preferably 80° C. or lower, yet more preferably 50° C. or lower, and particularly preferably 40° C. or lower.

The temperature for mixing the (a) component and the (b) component being 80° C. or lower means that an inner temperature (an internal temperature) of the mixture is 80° C. or lower. When the mixture is heated using an external device, such as an oil bath, a hot plate, and a mixer equipped with a thermal control function, the temperature not particularly limited as long as the temperature of the mixture itself is 80° C. or lower. Specifically, there is no problem even if a set temperature of the external device greater than 80° C.

A temperature for mixing the base polymer with the mixture obtained by mixing the (a) 3-[(3R*,4R*)-3-(dimethylamionomethyl)tetrahydropyran-4-yl]phenol and the (b) polar functional group-containing organic compound is not particularly limited, and may be appropriately selected in accordance with the intended purpose. The temperature is preferably 80° C. or lower, more preferably 70° C. or lower, yet more preferably 60° C. or lower, particularly preferably 50° C. or lower, and most preferably 40° C. or lower.

The temperature for mixing the base polymer with the mixture obtained by mixing the (a) component and the (b) component is also as described above, and there is no problem even if a set temperature of an external device is greater than 80° C.

A temperature for mixing the plasticizer with the mixture obtained by mixing the (a) 3-[(3R*,4R*)-3-(dimethylamionomethyl)tetrahydropyran-4-yl]phenol and the (b) polar functional group-containing organic compound is not particularly limited, and may be appropriately selected in accordance with the intended purpose. The temperature is preferably 80° C. or lower, more preferably 70° C. or lower, yet more preferably 60° C. or lower, particularly preferably 50° C. or lower, and most preferably 40° C. or lower. A temperature for mixing the resulting mixture with the base polymer is not particularly limited, and may be appropriately selected in accordance with the intended purpose. The temperature is preferably 80° C. or lower, more preferably 70° C. or lower, more preferably 60° C. or lower, particularly preferably 50° C. or lower, and most preferably 40° C. or lower.

The temperature for mixing the plasticizer with the mixture obtained by the (a) component and the (b) component is also as described above, and there is no problem even if a set temperature of an external device is greater than 80° C.

The mixing is not particularly limited, and may be appropriately selected in accordance with the intended purpose. The mixing preferably includes mixing in the presence of a volatile solvent.

The volatile solvent is not particularly limited, and may be appropriately selected in accordance with the intended purpose. Examples of the volatile solvent include aromatic hydrocarbons (e.g., toluene and the like), alicyclic hydrocarbons (e.g., cyclohexane, methyl cyclohexane, and the like), aliphatic hydrocarbons (e.g., hexane, heptane, and the like), ethers (e.g., tetrahydrofuran, diethyl ether, t-butyl methyl ether, and the like), ketones (e.g., acetone, methyl ethyl ketone, methyl isobutyl ketone, and the like), alcohols (e.g., ethanol, propanol, butanol, and the like), acetic acid esters (e.g., ethyl acetate, propyl acetate, isopropyl acetate, butyl acetate, isobutyl acetate, and the like), and the like. The above-listed examples may be used alone or in combination.

Among the above-listed examples, considering excellent solubility of components constituting the adhesive layer, a single solvent or a mixture of two or more solvents selected from aromatic hydrocarbons (e.g., toluene and the like), alicyclic hydrocarbons (e.g., cyclohexane, methyl cyclohexane, and the like), and aliphatic hydrocarbons (e.g., hexane, heptane, and the like) is preferably used.

The base polymer may be mixed with the volatile solvent before mixing with a mixture obtained by mixing (a) 3-[(3R*,4R*)-3-(dimethylamionomethyl)tetrahydropyran-4-yl]phenol and a (b) polar functional group-containing organic compound, or before mixing with a mixture obtained by mixing a plasticizer with (a) 3-[(3R*,4R*)-3-(dimethylamionomethyl)tetrahydropyran-4-yl]phenol and a (b) polar functional group-containing organic compound.

A temperature for mixing the base polymer and the volatile solvent is not particularly limited, and may be appropriately selected in accordance with the intended purpose. The temperature is preferably 80° C. or lower.

When the tackifier is used, the tackifier may be added to the mixture of the base polymer and the volatile solvent.

### Other Steps

The above-mentioned other steps are not particularly limited, and may be appropriately selected in accordance with the intended purpose. Examples of the above-mentioned other steps include a step of coating after forming the adhesive layer forming step, a step of laminating a support and an adhesive layer, a step of laminating a release liner, and the like.

The support, the adhesive layer, and the release liner are as described in the (Patch).

The step of laminating the support and the adhesive layer and the step of laminating the release liner are as described in the (Production method of patch: first embodiment).

### Coating Step after Step of Forming Adhesive Layer

The coating of the coating liquid for forming an adhesive layer, which is obtained in the step of forming the adhesive layer, is performed. by applying the coating liquid to the support or the release liner by means of a commonly used coater, such as a roll coater, a die coater, a gravure roll coater, a reverse coater, a kiss roll coater, a dip roll coater, a bar coater, a knife coater, a spray coater, and the like.

Drying of the coating liquid is performed with heating, for example, preferably at a temperature of 40° C. or higher, and preferably at a temperature of 150° C. or lower, more preferably 100° C. or lower, yet more preferably 90° C. or lower, and particularly preferably 80° C. or lower. Among the above-listed examples, the heating is preferably performed at a temperature of 40° C. or higher and 150° C. or lower, more preferably 40° C. or higher and 100° C. or lower, yet more preferably 40° C. or higher and 90° C. or lower, and particularly preferably 40° C. or higher and 80° C. or lower. The drying temperature drying duration, and drying method are adjusted in accordance with a solvent for use or an amount of the solvent used.

### EXAMPLES

Examples of the present invention will be described hereinafter, but the examples shall not be construed as limiting the scope of the present invention in any way.

### Comparative Example 1-1

Each of components constituting an adhesive layer was weighed and collected according to the formulation (Compound 1: 10% by weight, a styrene-isoprene-styrene block copolymer: 20%, liquid paraffin: 40% by weight, rosin: 30% by weight) presented in Comparative Example 1-1 of Table 1 and disclosed in PTL 1 (International Patent Application Publication No. WO 2019/156074). Note that, a unit for a numerical value of each component presented in Table 1 is % by mass.

First, Compound 1 and toluene were mixed and stirred (internal temperature: 50° C. to 80° C.) to obtain a mixture A. In toluene, a mixture of a styrene-isoprene-styrene block copolymer and a styrene-isoprene block copolymer (5002, available from JSR Corporation) and a rosin ester (KE-311, available from ARAKAWA CHEMICAL INDUSTRIES, LTD.) were mixed and stirred (internal temperature: 30° C. to 60° C.) to dissolve. The resulting solution was added to the mixture A, and the resulting mixture was mixed and stirred (internal temperature: 50° C. to 80° C.). Moreover, liquid paraffin (KAYDOL, available from Sonneborn) was added, and the resulting mixture was mixed and stirred (internal temperature: 50° C. to 80° C.) to prepare a coating liquid for forming an adhesive layer.

The coating liquid was applied onto a polyethylene terephthalate (PET) film processed with silicone (release liner, FILMBYNA 75E-0010 DG2.5, available from. FUJIMORI KOGYO CO., LTD.) in a manner that a thickness of a dried adhesive layer was to be approximately 100 μm. After drying the coating liquid in an oven set at 80° C. for 30 minutes, a PET film (a support) was laminated onto the surface of the adhesive layer, to thereby obtain a patch.

In accordance with the evaluation methods below, the presence of precipitated crystals in the adhesive layer of the patch and presence of deposited crystals on the release liner were evaluated. The results are presented in Table 1.

### (Evaluation Method) Presence of Precipitated Crystals of Compound 1 in the Adhesive Layer

The presence of precipitated crystals of Compound 1 in the adhesive layer was evaluated in accordance with the following
Good: After storing the patch for 3 days or longer at 30° C. or lower, absence of precipitated crystals in the adhesive layer was confirmed by visual observation.
Not good: After storing the patch for 3 days or longer at 30° C. or lower, presence of precipitated crystals in the adhesive layer was confirmed by visual observation.

### (Evaluation Method) Presence of Deposited Crystals of Compound 1 on the Release Liner

The presence of crystals of Compound 1 deposited on the release liner was evaluated in accordance with the following criteria.
Good: After storing the patch for 3 days or longer at 30° C. or lower, absence of precipitated crystals in the adhesive layer and absence of deposited crystals on the release liner were confirmed by visual observation.
Not good: After storing the patch for 3 days or longer at 30° C. or lower, presence of precipitated crystals in the adhesive layer and presence of deposited crystals on the release liner were confirmed by visual observation.

TABLE 1

| | | Comp. | Comp. | Comp. |
|---|---|---|---|---|
| | | Ex. 1-1 | Ex. 1-2 | Ex. 1-3 |
| Active | Compound 1 | 10 | 10 | 2 |
| ingredient | | | | |
| Base | SIS (5002) | 20 | 20 | 20 |
| Plasticizer | Liquid | 40 | 40 | 48 |
| | paraffin | | | |
| | (KAYDOL) | | | |
| Organic acid | Capric acid | NA | NA | NA |
| Amide | N-methyl | NA | NA | NA |
| | pyrrolidone | | | |
| Alcohol | Benzyl | NA | NA | NA |
| | alcohol | | | |
| Tackifier | Rosin ester | 30 | 30 | 30 |
| | (KE-311) | | | |

| | | | |
|---|---|---|---|
| Release liner | DG | BD | BD |
| Presence of precipitated | Not good | Not good | Not good |
| crystals | | | |
| Presence of deposited | Not good | Not good | Not good |
| crystals on release liner | | | |

### Comparative Example 1-2

A patch was produced and the presence of precipitated crystals in the adhesive layer and the presence of the deposited crystals on the release liner were evaluated in the same manner as in Comparative Example 1-1, except that “FILMBYNA 75E-0010 BD” available from FUJIMORI KOGYO CO., LTD. was used as the release liner. The results are presented in Table 1.

### Comparative Example 1-3

A patch was produced and the presence of the precipitated crystals in the adhesive layer and the presence of the deposited crystals on the release liner were evaluated in the same manner as in Comparative Example 1-2, except that components constituting an adhesive layer were weighed and collected according to the formulation of Comparative Example 1-3 of Table 1, the application of the coating liquid was adjusted so that a thickness of a dried adhesive layer was to be approximately 500 μm to match with the amount of Compound 1 the adhesive layer of Comparative Example 1-2, and the drying conditions of the coating liquid for forming an adhesive layer were changed to 80° C. for 50 minutes. The results are presented in Table 1.

It was confirmed from the results presented in Table 1 that the precipitation of crystals in the adhesive layer, and the deposition of the crystals on the release liner were observed in the patch of Comparative Example 1-1 that was produced according to the formulation disclosed in PTL 1, the patch of Comparative Example 1-2 in which a type of the release linear for use was changed from Comparative Example 1-1, and the patch of Comparative Example 1-3, in which the concentration of Compound 1 was changed to 2% by mass from Comparative Example 1-1.

It was made clear that the crystals were removed from the adhesive layer in the amount enough to deposit on the release liner even when the concentration of the drug in the adhesive layer was reduced. It was assumed that a formulation of an adhesive layer needs to be fundamentally revised.

### Example 1-1

A patch was produced and the presence of precipitated crystals in the adhesive layer and the presence of the deposited crystals on the release liner were evaluated in the same manner as in Comparative Example 1-3, except that the mixing and stirring of Compound 1 and the toluene was changed to mixing and stirring Compound 1 and capric acid in toluene (internal temperature: 50° C. to 50° C.) according to the formulation presented in Example 1-1 of Table 2. The results are presented in Table 2.

TABLE 2

| | | | | | Comp. | Comp. |
|---|---|---|---|---|---|---|
| | | | | | Ex. | Ex. |
| | | Ex. 1-1 | Ex. 1-2 | Ex. 1-3 | 1-4 | 1-5 |
| Active | Compound 1 | 2 | 2 | 2 | 2 | 2 |
| ingredient | | | | | | |
| Base | SIS (5002) | 20 | 20 | 20 | 20 | 20 |
| Plasticizer | Liquid | 44 | 44 | 44 | 44 | 44 |
| | paraffin | | | | | |
| | (KAYDOL) | | | | | |
| Organic | Capric acid | 4 | NA | NA | NA | NA |
| acid | | | | | | |
| Amide | N-methyl | NA | 4 | NA | NA | NA |
| | pyrrolidone | | | | | |
| Alcohol | Benzyl | NA | NA | 4 | NA | NA |
| | alcohol | | | | | |
| Ester | Isopropyl | NA | NA | NA | 4 | NA |
| | myristate | | | | | |
| Ether | Dimethyl | NA | NA | NA | NA | 4 |
| | isosorbide | | | | | |
| Tackifier | Rosin ester | 30 | 30 | 30 | 30 | 30 |
| | (KE-311) | | | | | |

| | | | | | |
|---|---|---|---|---|---|
| Release liner | BD | BD | BD | BD | BD |
| Presence of precipitated | Good | Good | Good | Not | Not |
| crystals | | | | good | good |
| Presence of deposited | Good | Good | Good | Good | Good |
| crystals on release liner | | | | | |

### Examples 1-2 to 1-3 and Comparative Examples 1-4 to 1-5

A patch was produced and the presence of the precipitated crystals in the adhesive layer and the presence of the deposited crystals on the release liner were evaluated in the same manner as in Example 1-1, except that components constituting an adhesive layer were weighed and collected according to the formulation of each of Examples 1-2 to 1-3 and Comparative Examples 1-4 to 1-5 of Table 2. The results are presented in Table 2.

According to the results presented in Table 2, the precipitated crystals in the adhesive layer of the patch and the deposited crystals on the release liner were not observed with the patch of Example 1-1, in which capric acid serving as an organic acid was added to the adhesive layer, the patch of Example 1-2, in which N-methyl pyrrolidone serving as an amide was added to the adhesive layer, and the patch of Example 1-3, in which benzyl alcohol serving as an alcohol was added to the adhesive layer.

Conversely, the precipitated crystals in the adhesive layer of the patch were observed with the patch of Comparative Example 1-4, in which isopropyl myristate serving as an eater was added to the adhesive layer, and the patch of Comparative Example 1-5, in which dimethyl isosorbide serving as an ether was added to the adhesive layer, even though the deposition of the crystals on the release liner was not observed.

### Example 1-4

A patch was produced and the presence of the precipitated crystals in the adhesive layer and the presence of the deposited crystals on the release liner were evaluated in the same manner as in Comparative Example 1-3, except that the mixing and stirring of Compound 1 and the toluene was changed mixing and stirring Compound 1, capric acid, crotamiton, alkyl lactate, and benzyl alcohol in toluene (internal temperature: 20° C. to 50° C.) according to the formulation presented in Example 1-4 of Table 3. The results are presented in Table 3.

TABLE 3

| | | | | | | | Comp. |
|---|---|---|---|---|---|---|---|
| | | Ex. | Ex. | Ex. | Ex. | Ex. | Ex. |
| | | 1-4 | 1-5 | 1-6 | 1-7 | 1-8 | 1-6 |
| Active | Compound 1 | 2 | 2 | 2 | 2 | 2 | 2 |
| ingredient | | | | | | | |
| Base | SIS (5505) | 30 | 30 | 30 | 30 | 30 | 30 |
| Plasticizer | Octyldodecyl | 26.5 | 28.5 | 30.5 | 30.5 | 29.5 | 38 |
| | myristate | | | | | | |
| Organic | Capric acid | 1.5 | 1.5 | 1.5 | 1.5 | NA | NA |
| acid | Oleic acid | NA | NA | NA | NA | 2.5 | NA |
| Amide | Crotamiton | 2 | NA | 2 | 2 | 2 | NA |
| Alcohol | Alkyl lactate | 4 | 4 | 4 | NA | NA | NA |
| | Benzyl | 4 | 4 | NA | NA | NA | NA |
| | alcohol | | | | | | |
| | Propylene | NA | NA | NA | 4 | 4 | NA |
| | glycol | | | | | | |
| | monocaprylate | | | | | | |
| Tackifier | Rosin ester | 30 | 30 | 30 | 30 | 30 | 30 |
| | (KE-311) | | | | | | |

| | | | | | | |
|---|---|---|---|---|---|---|
| Release liner | BD | BD | BD | BD | BD | BD |
| Presence of precipitated | Good | Good | Good | Good | Good | Not |
| crystals | | | | | | good |
| Presence of deposited | Good | Good | Good | Good | Good | Not |
| crystals on release liner | | | | | | good |

### Examples 1-5 to 1-8

A patch was produced and the presence of the precipitated crystals in the adhesive layer and the presence of the deposited crystals on the release liner were evaluated in the manner as in Example 1-4, except that components constituting an adhesive layer were weighed and collected according to the formulation of each of Examples 1-5 to 1-8 of Table 3. The results are presented in Table 3.

### Comparative Example 1-6

A patch was produced and the presence of the precipitated crystals in the adhesive layer and the presence of the deposited crystals on the release liner were evaluated in the same manner as in Comparative Example 1-3, except that components constituting an adhesive layer were weighed and collected according to the formulation of Comparative Example 1-6 of Table 3. The results are presented in Table 3.

According to the results presented in Table 3, the precipitated crystals in the adhesive layer of the patch and the deposited crystals on the release liner were not observed with the patches of Examples 1-4 to 1-8, in which at least one selected from the group consisting of organic acids, amides, and alcohols was added to the adhesive layer.

Conversely, the precipitated crystals in the adhesive layer of the patch and the deposited crystals on the release liner were observed with the patch of Comparative Example 1-6, in which at least one selected from the group consisting of organic acids, amides, and alcohols was not added to the adhesive layer.

### Example 1-9

A patch was produced and the presence of the precipitated crystals in the adhesive layer and the presence of the deposited crystals on the release liner were evaluated in the same manner as in Example 1-4, except that components constituting the adhesive layer were weighed and collected according to the formulation of Example 1-9 of Table 4 and the application of the coating liquid was adjusted so that a thickness of a dried adhesive layer was to be approximately 100 μm. The results are presented in Table 4.

### Example 1-10

A patch was produced and the presence of the precipitated crystals in the adhesive layer and the presence of the deposited crystals on the release liner were evaluated in the same manner as in Example 1-9, except that components constituting the adhesive layer were weighed and collected according to the formulation of Example 1-10 of Table 4, the application of the coating liquid for forming an adhesive layer was adjusted so that a thickness of a dried adhesive layer was to be approximately 500 μm, and the drying conditions of the coating liquid for forming an adhesive layer were changed to 80° C. for 50 minutes. The results are presented in Table 4.

TABLE 4

| | | Ex. 1-9 | Ex. 1-10 |
|---|---|---|---|
| Active | Compound 1 | 10 | 2 |
| ingredient | | | |
| Base | SIS (5002) | 20 | 20 |
| Organic acid | Capric acid | 1.5 | 1.5 |
| | Isostearic | 35 | 35 |
| | acid | | |
| Plasticizer | Octyldodecyl | 3.5 | 11.5 |
| | myristate | | |
| Tackifier | Rosin ester | 30 | 30 |
| | (KE-311) | | |

| | | |
|---|---|---|
| Release liner | BD | BD |
| Presence of precipitated | Good | Good |
| crystals | | |
| Presence of deposited | Good | Good |
| crystals on release liner | | |

According to the results presented in Table 4, the precipitated crystals in the adhesive layer of the patch and the deposited crystals on the release liner were not observed with the patches of Examples 1-9 to 1-10, in which capric acid and isostearic acid were added to the adhesive layer as organic acids.

### Reference Experiment 1

A patch was produced in the same manner as in Example 1-10, except that components for constituting the adhesive layer were weighed and collected according to the formulation of Reference Experiment 1 of Table 5.

The presence of the precipitated crystals in the adhesive layer of the patch and the presence of the deposited crystals on the release liner were evaluated under conditions of a high temperature and high humidity (40° C./75% RH).

After storing the patch for one day in the high temperature and high humidity atmosphere (40° C./75% RH), it was confirmed through visual observation that there was no precipitation of crystals in the adhesive layer.

After storing the patch for one day in the high temperature and high humidity atmosphere (40° C./75% RH), moreover, it was confirmed through visual observation that there was no precipitation of crystals in the adhesive layer, nor deposition of crystals on the release liner.

TABLE 5

| | | Reference | Reference |
|---|---|---|---|
| | | Experiment 1 | Experiment 2 |
| Active | Compound 1 | 2.0 | 2.0 |
| ingredient | | | |
| Base | SIS (Quintac | 30.0 | 30.0 |
| | 3520) | | |
| Plasticizer | Liquid | 47.0 | 47.0 |
| | paraffin | | |
| | (Hydrobrite | | |
| | HV) | | |
| Amide | Crotamiton | NA | 2.0 |
| Alcohol | Benzyl | NA | 4.0 |
| | alcohol | | |
| Organic acid | Oleic acid | 6.0 | NA |
| Tackifier | Rosin ester | 15.0 | 15.0 |
| | (KE-311) | | |

### Reference Experiment 2

A patch was produced in the same manner as in Example 1-10, except that components for constituting the adhesive layer were weighed and collected according to the formulation of Reference Experiment 2 of Table 5.

The presence of the precipitated crystals in the adhesive layer of the patch and the presence of the deposited crystals on the release liner were evaluated at conditions of a high temperature and high humidity (40° C./75% RH).

After storing the patch for one day in the high temperature and high humidity atmosphere (40° C./75% RH), precipitation of crystals in the adhesive layer was confirmed through visual observation.

After storing the patch for one day in the high temperature and high humidity atmosphere (40° C./75% RH), moreover, it was co firmed through visual observation that there was no precipitation of crystals in the adhesive layer, nor deposition of crystals on the release liner.

According to the results of reference Experiments 1 and 2, under the storage conditions of a high temperature and high humidity (40° C./75% RH) for one day, precipitation of crystals in the adhesive layer of the patch was not observed with the patch of Reference Example 1, in which oleic acid was added to the adhesive layer as an organic acid, but precipitation of crystals was confirmed with the patch of Reference Example 2, in which an organic acid is not added, and crotamiton serving as an amide and benzyl alcohol serving as an alcohol were added to the adhesive layer.

### Reference Experiment 3

A patch was produced in the same manner as in Example 1-10, except that components for constituting the adhesive layer were weighed and collected according to the formulation of Reference Experiment 3 of Table 6.

The presence of the precipitated crystals in the adhesive layer of the patch and the presence of the deposited crystals on the release liner were evaluated at conditions of a high temperature and high humidity (40° C./75% RH).

After storing the patch for 14 days in the high temperature and high humidity atmosphere (40° C./75% RH), it was confirmed through visual observation that there was no precipitation of crystals in the adhesive layer.

After storing the patch for 14 days in the high temperature and high humidity atmosphere (40° C./75% RH), moreover, it was confirmed through visual observation that there was no precipitation of crystals in the adhesive layer, nor deposition of crystals on the release liner.

TABLE 6

| | | Reference | Reference |
|---|---|---|---|
| | | Experiment 3 | Experiment 4 |
| Active | Compound 1 | 2.0 | 2.0 |
| ingredient | | | |
| Base | SIS (5505) | 35.0 | 35.0 |
| Plasticizer | Octyldodecyl | 22.2 | 24.5 |
| | myristate | | |
| Amide | Crotamiton | 2.0 | 2.0 |
| Alcohol | Propylene | 4.0 | 4.0 |
| | glycol | | |
| | monocaprylate | | |
| Organic acid | Oleic acid | 4.8 | 2.5 |
| Tackifier | Rosin ester | 30.0 | 30.0 |
| | (KE-311) | | |

### Reference Experiment 4

A patch was produced in the same manner as in Example 1-10, except that components for constituting the adhesive layer were weighed and collected according to the formulation of Reference Experiment 4 of Table 6.

The presence of the precipitated crystals in the adhesive layer of the patch and the presence of the deposited crystals on the release liner were evaluated at conditions of a high temperature and high humidity (40° C./75% RH).

After storing the patch for 14 days in the high temperature and high humidity atmosphere (40° C./75% RH), precipitation of crystals in the adhesive layer was confirmed by visual observation.

After storing the patch for 14 days in the high temperature and high humidity atmosphere (40° C./75% RH), moreover, it was confirmed through visual observation that there was no precipitation of crystals in the adhesive layer, nor deposition of crystals on the release liner.

According to the results of Reference Experiments 3 and 4, under the storage conditions of the high temperature and high humidity (40° C./75% RH) for 14 days, precipitation of crystals in the adhesive layer of the patch was not observed with the patch of Reference Example 3, in which oleic acid serving as an organic acid was added in the amount of 2 molar equivalents relative to Compound 1, but precipitation of crystals was confirmed with the patch of Reference Example 4, in which oleic acid was added only in the amount of 1 molar equivalent relative to Compound 1.

### Test Example 1: Evaluation of Analgesic Effect in Tail Flick Test of Hairless Rat

In the tail flick test, 10-week old hairless rat (HWY/Slc, available from Japan SLC, Inc.) were grouped into a patch applied group and an untreated group (two rats in each group). To each of the rats in the patch applied group, the patch of Example 1-4 (the drug content: 5.3 mcg) was applied. To each of the rats in the untreated group, the patch was not applied.

Eighteen hours after the application of the patch, heat laser was applied to a tail of each of the hairless rats of both groups by means of a tail flick unit—an analgesic effect measuring device (available from Ugo Basile SRL) to measure a reaction time. The reaction time was a time period between the starting point of the irradiation and a point when the tail moved. The results are presented in Table 7.

It was judged that the longer the reaction time was, the better the analgesic effect exhibited.

TABLE 7

| | Average reaction time |
|---|---|
| Patch applied group | 16.0 seconds |
| Untreated group | 10.5 seconds |

According to the results of Table 7, the reaction time of the patch group was longer than the reaction time of the untreated group, and the expression of the analgesic effect of the patch was confirmed. Moreover, significant skin irritation (e.g., redness) was not observed on the area of the hairless rats where the patch was applied in the patch applied group, and side effects (e.g., abnormal behaviors) were not also observed.

### Comparative Example 2-1

Components for constituting an adhesive layer were weighed and collected according to the formulation (Compound 1: 10% by weight, styrene-isoprene-styrene block copolymer: 20%, liquid paraffin: 40% by weight, rosin: 30% by weight) of Comparative Example 2-1 presented in Table 8 and disclosed in PTL 1 (International Patent Application Publication No. WO 2019/156074). Note that, a unit for a numerical value of each component depicted in Table 8 is % by mass.

First, Compound 1 and toluene were mixed (internal temperature: 50° C. to 80° C.) to obtain a mixture A. In toluene, a mixture of a styrene-isoprene-styrene block copolymer and a styrene-isoprene block copolymer (5002, available from JSR Corporation) and a rosin ester (KE-311, available, from ARAKAWA CHEMICAL INDUSTRIES, LTD.) were mixed and stirred (internal temperature: 30° C. to 60° C.) to dissolve. The resulting solution was added to the mixture A, and the resulting mixture was mixed and stirred (internal temperature: 50° C. to 80° C.). Moreover, liquid paraffin (KAYDOL, available from Sonneborn) was added and the resulting mixture was mixed and stirred (internal temperature: 50° C. to 80° C.) to prepare a coating liquid for forming an adhesive layer.

The coating liquid was applied onto a polyethylene terephthalate (PET) film processed with silicone (release liner, FILMBYNA 75E-0010 ED, available from FUJIMORI KOGYO CO., LTD.) in a manner that a thickness of a dried adhesive layer was to be approximately 100 μm. After drying the coating liquid in an oven set at 80° C. for 30 minutes, a PET film (i.e., a support) was laminated onto the surface of the adhesive layer, to thereby obtain a patch.

Fulfillment of production of the patch, and adhesive force and cohesive force of the adhesive layer of the patch were evaluated according to the following evaluation methods. The results is are presented in Table 8.

### (Evaluation Method) Fulfillment of Production of Patch

The practicability of production of the patch was evaluated according to the following criteria.
Good: The dissolution of Compound 1 confirmed during the production process by visual observation, and a patch was produced without any problem.
Not good: Compound 1 did not dissolve during the production process, and a uniformly patch could not be produced.

### (Evaluation Method) Adhesion Force

The adhesion force of the adhesive layer of the patch was evaluated based on the feel when a finger was released from the adhesive layer after pressing the finger onto the adhesive layer of the patch for 1 second or longer, according to the following criteria.
Good: The adhesion force equal to or greater than Tulobuterol patch known in the art was felt.
Fair: The adhesion force was relatively weak, but it was not problematic for practical use.
Not good: The adhesion force was weak and it was not suitable for practical use.

### (Evaluation Method) Cohesive Force

The cohesive force of the adhesive layer of the patch was evaluated according to the following criteria.
Good: No adhesive residue was observed.
Fair: The cohesive force was rather inadequate, but it was not problematic.
Not good: Adhesive residues, collapse of the shape, etc. were observed, and the cohesive force was significantly inadequate.

TABLE 8

| | | Comp. | | | |
|---|---|---|---|---|---|
| | | Ex. 2-1 | Ex. 2-1 | Ex. 2-2 | Ex. 2-3 |
| Active | Compound 1 | 10 | 2 | 2 | 2 |
| ingredient | | | | | |
| Base | SIS (5002) | 20 | 20 | 20 | 20 |
| Plasticizer | Liquid | 40 | 43 | 43 | 43 |
| | paraffin | | | | |
| | (KAYDOL) | | | | |
| Organic acid | Capric acid | NA | 5 | NA | NA |
| Amide | N-methyl | NA | NA | 5 | NA |
| | pyrrolidone | | | | |
| Alcohol | Benzyl | NA | NA | NA | 5 |
| | alcohol | | | | |
| Tackifier | Rosin ester | 30 | 30 | 30 | 30 |
| | (KE-311) | | | | |

| Fulfillment of production | Not good | Good | Good | Good |
|---|---|---|---|---|
| of patch | | | | |
| Adhesion force | Not good | Fair to | Fair to | Fair to |
| | | Good | Good | Good |
| Cohesive force | Good | Good | Good | Good |

### Example 2-1

A patch was produced, and fulfillment of production of the patch and adhesion force and cohesive force of the adhesive layer of the patch were evaluated in the same manner as in Comparative Example 2-1, except that the mixing Compound 1 and toluene was changed to mixing and stirring Compound 1 and capric acid in toluene (internal temperature: 50° C. to 80° C.) according to the formulation of Example 2-1 of Table 8, the application of the coating liquid was adjusted so that a thickness of a dried adhesive layer was to be approximately 500 μm, and the drying conditions of the coating layer were changed to at 80° C. for 50 minutes. The results are presented in Table 8.

### Examples 2-2 to 2-3

A patch was produced, a d fulfillment of production of the patch and adhesion force and cohesive force of the adhesive layer of the patch were evaluated in the same manner as in Example 2-1, except that components for constituting the adhesive layer were weighed and collected according to the formulation of each of Examples 2-2 to 2-8 presented in Table 8. The results are presented in Table 8.

According to the results of Table 8, in Comparative Example 2-1 where the production was performed according to the formulation disclosed in PTL 1, Compound 1 did not dissolve during the production process and a coating liquid for an adhesive layer, in which Compound 1 was completely dissolved, could not be produced. As the coating liquid was used to produce a patch regardless of the above-described defect, moreover, a patch that was not suited for practical use was produced. The produced patch had the heterogeneous adhesive layer (the Compound 1 content varied depending on the location of the adhesive layer). In addition, the adhesion force of the adhesive layer of the patch was weak.

Meanwhile, it was confirmed with Examples 2-1 to 2-3, in which an adhesive layer was formed using the mixture obtained by mixing Compound 1 and the polar functional group-containing organic compound at 80° C. or lower, that dissolution of Compound 1 was confirmed during the production process by visual observation, a patch could be produced without any problem, and there was no problem with the adhesion force of the adhesive layer of the patch.

### Example 2-4

A patch was produced, and fulfillment of production of the patch and adhesion force and cohesive force of the adhesive layer of the patch were evaluated in the same manner as in Example 2-1, except that the mixing and stirring Compound 1 and capric acid in toluene was changed to mixing and stirring Compound 1, crotamiton, alkyl lactate, and benzyl alcohol in toluene (internal temperature: 50° C. to 30° C.) according to the formulation of Example 2-4 of Table 9. The results are presented in Table 9.

TABLE 9

| | | Ex. 2-4 | Ex. 2-5 | Ex. 2-6 |
|---|---|---|---|---|
| Active | Compound 1 | 2 | 2 | 2 |
| ingredient | | | | |
| Base | SIS (Quintac | 30 | 30 | 30 |
| | 3520) | | | |
| Plasticizer | Liquid | 43 | 41.5 | 40.5 |
| | paraffin | | | |
| | (Hydrobrite | | | |
| | HV) | | | |
| Organic acid | Capric acid | NA | 1.5 | NA |
| | Oleic acid | NA | NA | 2.5 |
| Amide | Crotamiton | 2 | 2 | 2 |
| Alcohol | Alkyl | 4 | 4 | 4 |
| | lactate | | | |
| | Benzyl | 4 | 4 | 4 |
| | alcohol | | | |
| Tackifier | Terpene | 15 | 15 | 15 |
| | resin | | | |
| | (PX1150) | | | |

| | | | |
|---|---|---|---|
| Fulfillment of production | Good | Good | Good |
| of patch | | | |
| Adhesion force | Good | Fair | Fair |
| Cohesive force | Good | Good | Good |

### Examples 2-5 to 2-6

A patch was produced, and fulfillment of production of the patch and adhesion force and cohesive force of the adhesive layer of the patch were evaluated in the same manner as in Example 2-1, except that the mixing and stirring of Compound 1 and the capric acid in the toluene was changed o mixing and stirring Compound 1, capric acid or oleic acid, crotamiton, alkyl lactate, and benzyl alcohol in toluene (internal temperature: 20° C. to 50° C. ) according to the formulation of each of Examples 2-5 to 2-6 of Table 9. The results are presented in Table 9.

It was confirmed from the results of Table 9 that, with the patches of Examples 2-4 to 2-5, in which an adhesive layer was formed using the mixture obtained by mixing Compound 1 and the polar functional group-containing organic compound at 80° C. or lower, dissolution of Compound 1 was confirmed during the production process by visual observation, a patch could be produced without problem, and there was no problem with an adhesion force of the adhesive layer of the patch.

### Examples 2-7 to 2-8

A patch was produced, and fulfillment of production of the patch and adhesion force and cohesive force of the adhesive layer of the patch were evaluated in the same manner as in Example 2-4, except that components for constituting the adhesive layer were weighed and collected according to the formulation of each of Examples 2-7 to 2-0 presented in Table 10. The results are presented in Table 10.

TABLE 10

| | | Ex. 2-7 | Ex. 2-8 |
|---|---|---|---|
| Active | Compound 1 | 2 | 2 |
| ingredient | | | |
| Base | SIS (5505) | 35 | 40 |
| Plasticizer | Octyldodecyl | 24.5 | 19.5 |
| | myristate | | |
| Organic acid | Capric acid | 2.5 | 2.5 |
| Amide | Crotamiton | 2 | 2 |
| Alcohol | Propylene | 4 | 4 |
| | glycol | | |
| | monocaprylate | | |
| Tackifier | Rosin ester | 30 | 30 |
| | (KE-311) | | |

| | | |
|---|---|---|
| Fulfillment of production | Good | Good |
| of patch | | |
| Adhesion force | Good | Good |
| Cohesive force | Good | Good |

It was confirmed from the results of Table 10 that, with the patches of Examples 2-7 to 2-8, in which an adhesive layer was formed using the mixture obtained by mixing Compound 1 and the polar functional group-containing organic compound at 80° C. or lower, dissolution of Compound 1 was confirmed during the production process by visual observation, a patch could be produced without any problem, and there was no problem with the adhesion force of the adhesive layer of the patch.

For example, the present invention includes the following embodiments.
<1> A patch including:
   an adhesive layer,
wherein the adhesive layer includes:
   (a) 3-[(3R*,4R*)-3-(dimethylamionomethyl)tetrahydropyran-4-yl]phenol; and
   (b) at least one selected from the group consisting of organic acids, amides, and alcohols, and
wherein an amount of the (b) is 50 parts by mass or greater relative to 100 parts by mass of the (a).
<2> The patch according to <1>,
   wherein the organic acids are fatty acids.
<3> The patch according to <1> or <2>,
   wherein an amount of the (a) is 10% by mass or less relative to a total amount of the adhesive layer.
<4> The patch according to any one of <1> to <3>,
   wherein the adhesive layer further includes a thermoplastic elastomer.
<5> The patch according to <4>,
   wherein the thermoplastic elastomer includes a styrene-based block copolymer.
<6> The patch according to <5>,
   wherein the styrene-based block copolymer includes a mixture of a styrene-isoprene-styrene block copolymer and a styrene-isoprene block copolymer.
<7> The patch according to any one of <1> to <6> ,
   wherein the patch is for use in a treatment, or prevention, or both a treatment and prevention of chronic pains.
<8> A production method of a patch according to any one of <1> to <7>, including:
a mixing step that includes mixing 50 parts by mass or greater of the (b) at least one selected from the group consisting of organic acids, amides, and alcohols with 100 parts by mass of the (a) 3-[(3R*,4R*)-3-(dimethylamionomethyl)tetrahydropyran-4-yl]phenol.
<9> The production method according to <8>,
   wherein the mixing step is carried out in the presence of a volatile solvent.
<10> The production method according to <8> or <9>,
   wherein the mixing is mixing the (a) 3-[(3R*,4R*)-3-(dimethylamionomethyl)tetrahydropyran-4-yl]phenol with the (b) at least one selected from the group consisting of organic acids, amides, and alcohols at 80° C. or lower.
<11> The production method according to any one of <8> to <10>, further including:
a step that includes mixing, at 80° C. or lower, a thermoplastic elastomer with a mixture obtained by mixing the (a) 3-[(3R*,4R*)-3-(dimethylamionomethyl)tetrahydropyran-4-yl]phenol and the (b) at least one selected from the group consisting of organic acids, amides, and alcohols.
<12> A production method of a patch, including:
a step of forming an adhesive layer with a mixture,
wherein the mixture is obtained by mixing (a) 3-[(3R*,4R*)-3-(dimethylamionomethyl)tetrahydropyran-4-yl]phenol, and (b) a polar functional group-containing organic compound at 80° C. or lower.
<13> The production method according to <12>,
   wherein the step of forming the adhesive layer further includes mixing a base polymer with the mixture at 80° C. or lower.
<14> The production method according to <12> or <13>,
   wherein the step of forming the adhesive layer includes forming the mixture in the presence of a volatile solvent.
<15> The production method according to any one of <12> to <14>,
   wherein the polar functional group-containing organic compound includes at least one selected from the group consisting of organic acids, amides, and alcohols.

1. A patch comprising:
an adhesive layer,
wherein the adhesive layer includes:
(a) 3-[(3R*,4R*)-3-(dimethylamionomethyl)tetrahydropyran-4-yl]phenol; and
(b) at least one selected from the group consisting of organic acids, amides, and alcohols, and
wherein an amount of the (b) is 50 parts by mass or greater relative to 100 parts by mass of the (a).

2. The patch according to claim 1,
wherein the organic acids are fatty acids.

3. The patch according to claim 1,
wherein an amount of the (a) is 10% by mass or less relative to a total amount of the adhesive layer.

4. The patch according to claim 1,
wherein the adhesive layer further includes a thermoplastic elastomer.

5. The patch according to claim 4,
wherein the thermoplastic elastomer includes a styrene-based block copolymer.

6. The patch according to claim 5,
wherein the styrene-based block copolymer includes a mixture of a styrene-isoprene-styrene block copolymer and a styrene-isoprene block copolymer.

7. The patch according to claim 1,
wherein the patch is for use in a treatment, or prevention, or both a treatment and prevention of chronic pains.

8. A production method of a patch according to claim 1, comprising:
a mixing step that includes mixing 50 parts by mass or greater of the (b) at least one selected from the group consisting of organic acids, amides, and alcohols with 100 parts by mass of the (a) 3-[(3R*,4R*)-3-(dimethylaminomethyl)tetrahydropyran-4-yl]phenol.

9. The production method according to claim 8,
wherein the mixing step is carried out in the presence of a volatile solvent.

10. The production method according to claim 8,
wherein the mixing is mixing the (a) 3-[(3R*,4R*)-3-(dimethylaminomethyl)tetrahydropyran-4-yl]phenol with the (b) at least one selected from the group consisting of organic acids, amides, and alcohols at 80° C. or lower.

11. The production method according to claim 8, further comprising:
a step that includes mixing, at 80° C. or lower, a thermoplastic elastomer with a mixture obtained by mixing the (a) 3-[(3R*,4R*)-3-(dimethylaminomethyl)tetrahydropyran4-yl]phenol and the (b) at least one selected from the group consisting of organic acids, amides, and alcohols.

12. A production method of a patch, comprising:
a step of forming an adhesive layer with a mixture,
wherein the mixture is obtained by mixing (a) 3-[(3R*,4R*)-3-(dimethylaminomethyl)tetrahydropyran-4-yl]phenol, and (b) a polar functional group-containing organic compound at 80° C. or lower.

13. The production method according to claim 12,
wherein the forming of forming the adhesive layer further includes mixing a base polymer with the mixture at 80° C. or lower.

14. The production method according to claim 12,
wherein the forming of forming the adhesive layer includes forming the mixture in the presence of a volatile solvent.

15. The production method according to claim 12,
wherein the polar functional group-containing organic compound includes at least one selected from the group consisting of organic acids, amides, and alcohols.
